(19) United States
(12) Patent Application Publication
(10) Pub. No.: US 2009/0311212 A1
(43) Pub. Date: Dec. 17, 2009

(54) Agent for Forming Double Eyelid

(75) Inventor: Yasunobu Horii, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(73) Assignee: Itsuo Futatsugi

(21) Appl. No.: 12/137,012

(22) Filed: Jun. 11, 2008

(51) Int. Cl. A61K 8/72; A61Q 99/00

(52) U.S. Cl. 424/78.04

(57) ABSTRACT

An agent for forming double eyelid is provided, which contains rubber latex, synthetic resin emulsion and alkanolamine therein. In such agent, those three component elements are admixed with one another, such that the mixing ratios (wt. %) respectively thereof fall within a polygonal region defined in ternary composition diagram shown in FIG. 1, wherein the polygonal region has five apexes A, B, C, D and E defined below relative to three coordinate axes x, y and z, wherein the x, y and z respectively relate to the three mixing ratios respectively of the rubber latex, synthetic resin emulsion and alkanolamine.
A (33.0, 64.0, 3.0)
B (49.0, 48.0, 3.0)
C (51.9, 48.0, 0.1)
D (34.9, 65.0, 0.1)
E (33.0, 65.0, 2.0)
   The agent so prepared is effective for forming double eyelid in an excessively taut top lid of human's eye.

### BACKGROUND OF THE INVENTION

1. Technical Field

The present invention relates to a chemical agent for providing a fold to a human's eyelid to thereby form a double eyelid therein. In particular, the invention is directed to such double eyelid forming agent which may be applied on a lower end region of user's top lid and left dry for solidification into a film secured thereon, thereby enabling the user to form a fold relative to such film, so that a double eyelid is formed in his or her top lid.

2. Description of Prior Art

At first, it is noted that the inventors of the present invention previously made this kind of double eyelid forming agent and filed a patent application therefor in Japan, which is assigned with the filing number: Japanese Patent Application No. Hei 1-3790. This Japanese patent application has been laid open with the publication number: Japanese Laid-Open Patent Publication No. Hei 2-188512, and eventually allowed and published under “Japanese Examined Publication No. Hei 6-62384”. Hereinafter, this prior art shall be referred to as “JP 6-62384”.

According to the JP 6-62384, the double eyelid forming agent is an eyelid treatment agent adapted to be directly applied on a lower edge region of human eye's top lid. Briefly stated, the agent is applied by a user on and along a lower end region of his or her top lid, and then left dry for solidification into a film secured thereon Thereafter, the user causes a fold to form relative to such film, so that a double eyelid is formed in his or her top lid.

Preparation of this double eyelid forming agent is characterized by admixing a rubber latex with one selected from the group of synthetic resin materials which consists of acrylic resins, urethan resins and vinyl resins. In this respect, according to the JP 6-62384, 85 to 50% by weight of the rubber latex may be admixed with 15 to 50% by weight of the afore-said one of synthetic resin materials.

In the above-described double eyelid forming agent, it is important that the rubber latex used should be of a property not exceeding a statutory permissible range set by regulations for hazardous materials that will adversely affect human body and natural environment. For example, one example of recommended latex is a low ammonium latex (containing approx. 0.25% of ammonia) with 0.8% or less of total alkali content therein, as prescribed by the code K 6381 of JIS (Japanese Industrial Standards).

Also, it is important that the synthetic resin materials used in the double eyelid forming agent be limited to appropriate synthetic resin materials to be admixed with the foregoing rubber latex, which fulfills the purposes of: (i) causing the rubber latex to get dry rapidly; (ii) improving water resistance and perspiration resistance (i.e. a property of resisting physiologic saline) which are required for the rubber latex to attain as a resulting dried film on human eyelid; and (iii) enhancing a hardness of that film.

Further, such synthetic resin material is required to have a good compatibility with above-defined rubber latex, so that the rubber latex mixed with the synthetic resin material becomes a film at minimum temperature of 20° C. or less. Also, the synthetic resin material is required to have a sufficient water resistance and a sufficient perspiration resistance. As indicated in the JP 6-62384, this sort of synthetic resin material may preferably be an emulsion type of synthetic resin material containing 38 to 55% by weight of solid content therein, which is available on the market. Typical example of such synthetic resin material includes: acrylic resins; urethan resins, or vinyl resins inclusive of vinyl chloride resins, styrene resins and vinyl acetate resins, for example. Of course, this is not imitative.

In practice, as taught in the JP 6-62384, an exemplary process for forming a double eyelid by means of the above-described agent is comprised of: a first step wherein a user closes his or her one eye or directs the same downwards by glancing down for instance, to thereby stretch and widen the top lid of the eye vertically; a second step wherein, while keeping the top lid stretched as such, 2 mm or less width of the agent is applied on and along the lower end region of that particular top lid, except as otherwise specified, by use of a brush or the like, and a third step of allowing the thus-applied agent to become dried. It is noted that, at that final drying step, the applied agent may be left as it is and become naturally dried under ambient temperature, or alternatively, the applied agent be forcibly dried by application of heat thereto for reducing the drying time.

Subsequently, the agent, thus applied and dried in the foregoing manner, is solidified into a film which is secured on the lower end region of top lid. Such film is provided with a certain hardness and an elastic property inherent in rubber content therein. Therefore, when the user opens the closed eye to cause contraction of the top lid, the non-applied region of that top lid, where the agent is not applied, is naturally contracted, whereas on the other hand, the applied region of the top lid, where the agent has been applied, is not contracted because of the thus-formed film of agent remaining hard and non-contractive against the contraction of eye lid. This causes formation of a fold in the top lid at a point above and along the film, so that a double eyelid is formed in the user's top lid.

From the description above, it is to be seen that the double lid forming agent is applied on the top lid, and then the agent is solidified into a film which is secured on that particular top lid. But, it is important that so formed film of the agent should not easily be flaked away from the top lid during an ordinary daily action of the user and that the presence of such film should not give the user any uncomfortable touch, such as a stretched state of his or her top lid. Hence, essential requirements for the film created in this double eyelid forming agent are: a firm adherence of the film to the top lid; an appropriate flexibility of rubber content for rendering the film stretchable and contractible responsive to the respective stretching and contracting actions of the top lid; and further, a certain hardness of the film sufficient to form and retain a double eyelid in the top lid; namely, a hardness which is not only greater than that of the top lid, but also has a rigidity to withstand a force applied to the film when a user bends the top lid relative to that film in the process of forming the double eyelid.

In this regard, required conditions of the film for achieving the above-noted properties are such hat a rate of stretchability of the film should fall in the ranges of from 300% to 500%, and a hardness of the film fall in the ranges of from 100 to 150 according to a hardness prescribed by the code K 6301 of JIS. Experiments indicate that any film created out of such ranges of stretchability and hardness did not cause any satisfied formation of double eyelid and did not stably retain a formed double eyelid, neither.

If the film simply has any high degree of hardness, regardless of the foregoing conditions, for example, the film itself formed on a skin of use's top lid will not be stretched and contracted in response to the respective stretching and contracting movements of the top lid, each time the user opens and closes his or her eyes. As a result thereof, for example, the film will be separated from the skin of user's top lid, or the top lid will remain in a stretched state due to such non-elastic state of the film, which makes the user feel uncomfortable and uneasy at the eyelid.

By contrast, let us assume that the film is merely soft and elastic due to an elasticity of rubber content therein. Otherwise stated, if an original agent for forming the film is composed solely of a rubber latex, the component element of the rubber latex is a natural rubber or a synthetic rubber, which has a small hardness of as low as 20 to 100 (under the code K 6301 of JIS). In that case, the hardness of the film is so insufficient that the film is easily deformed and stretched even by a weakly stretched state of the user's top lid at the time when the user normally opens his or her eyes, and therefore, it is impossible to form a double eyelid in the top lid.

Also, in some cases, the user applies an excessively increased amount of the double eyelid forming agent on his or her top lid in an attempt to create a thick layer of resulting film thereon. But, in that case, the user will become more uncomfortable or uneasy with such thick layer of film at the top lid. Further, since that thick layer of film protrudes from the skin of user's top lid, the user will need to apply excessive plural layers of cosmetics surrounding such protrudent film to compensate for a difference in level between the film and the surface of user's skin of top lid, so that a boundary between the film and user's skin becomes unclear and invisible. With those drawbacks in view, a recommended thickness of the double forming agent to be applied on the top lid is in the range of from 50μ to 5μ, although it may vary depending on a softness or elasticity of individual user's top lid.

As stated above, in applying the double eye forming agent, it is important that a thickness of the agent to be applied on the top lid should be controlled and reduced to a lowest possible degree. However, one more problem in the double eye forming agent arises from a hardness of user's top lid. Namely, in the case where the user's top lid is relatively hard, there may be no difference in hardness between such hard top lid and a resulting film of the agent formed on that particular top lid. In that case, the user will find it difficult to manually bend his or her top lid to form a desired double eyelid therein. Such problem may preferably be solved by using a pointed jig or piece as a auxiliary means for causing formation of double eyelid. Specifically, the user may press a suitable pointed piece against a local region of the top lid above the resulting film of agent, to thereby enable him or her to easily bend that local region of top lid, so that a fold is formed relative to that bent region and therefore a double eyelid is defined in this hardened state of top lid.

As an advantageous aspect of the double eyelid forming agent, a frequent or a long-term continued application of the agent on the top lid is effective in settling and leaving a foldable base in a given region of that top lid, wherein such settled foldable base is a point which a user can readily bend to form the afore-said fold in the top lid, hence defining a double eyelid therein. Thus, the settled foldable base remains firm and stable in the top lid, even when the agent is not applied thereto, thereby enabling the user to easily form and maintain a double eyelid at any time in a natural fashion. Accordingly, the present double eyelid forming agent may be used for cosmetic surgery as a safe and convenient cosmetic element.

While being provided with the foregoing various excellent properties, the double eyelid forming agent disclosed in the JP 6-62384 may not work well for an excessively taut skin surface of top lid in some cases, and in particular for a young user's excessively taut skin of top lid for instance. In other words, even after the double eyelid forming agent has been neatly applied on the lower end region of young user's top lid and left dry sufficiently, it may be the case that a resulting film of the agent will be inferior in tensile strength to such excessively high tautness of top lid skin and will be flaked away from the top lid, which renders it difficult to form double eyelid in the young user's eye. Hence, it has been desired to develop and realize an improved double eyelid forming agent that insures to form double eyelid reliably in the excessively taut skin of top lid.

### SUMMARY OF THE INVENTION

As mentioned above, there has been the problem that double eyelid may hardly be formed in an excessively taut skin of eye's top lid. It is therefore a purpose of the present invention to provide an improved agent for forming double eyelid which makes it possible to insure forming double eyelid even in such excessively taught skin of top lid.

In order to achieve the purpose, a double eyelid forming agent in accordance with the present invention is characterized, as a most significant aspect, by containing synthetic resin emulsion at an increased mixing ratio greater than the previously stated mixing ratio of synthetic resin emulsion in the JP 6-62384.

Namely, in accordance with the present invention, an agent for forming double eyelid is basically characterized by containing:
rubber latex;
synthetic resin emulsion; and
alkanolamine,
such rubber latex, synthetic resin emulsion and alkanolamine being admixed with one another, based on weight percent, such that mixing ratios respectively of the rubber latex, synthetic resin emulsion and alkanolamine fall within a polygonal region with five apexes A, B, C, D and E which is defined in a ternary composition diagram plotted with three coordinate axes x, y and z as shown in FIG. 1, wherein the x relates to mixing ratios (percent by weight) of the rubber latex, the y relates to mixing ratios (percent by weight) of the synthetic resin emulsion, and said z relates to mixing ratios (percent by weight) of alkanolamine, and wherein the five apexes A, B, C, D and E are defined as follows in relation to those x, y and z.
A (33.0, 64.0, 3.0)
B (49.0, 48.0, 3.0)
C (51.9, 48.0, 0.1)
D (34.9, 65.0, 0.1)
E (33.0, 65.0, 2.0)

Preferably, the mixing ratios respectively of the rubber latex, synthetic resin emulsion, and alkanolamine may fall within another polygonal region with five apexes F, G, H, I and J in the ternary composition diagram, wherein such apexes F, G, H, I and J are defined as follows in relation to the three coordinate axes x, y and z.
F (37.0, 61.0, 2.0)
G (49.0, 49.0, 2.0)
H (50.9, 49.0, 0.1)
I (37.0, 62.0, 1.0)
J (37.0, 62.0, 1.0)

As one preferred mode of the present invention, the afore-said synthetic resin emulsion may be one selected from the group consisting of: emulsion of acrylic ester resin; emulsion of resin of ester of methacrylic acid; emulsion of styrene resin; emulsion of styrene-acrylic copolymer resin; emulsion of styrene-methacrylic copolymer resin; emulsion of acrylamide-acrylamide copolymer resin; emulsion of modified ethylene-vinyl acetate copolymer resin; emulsion of ethylene-vinyl acetate copolymer resin; emulsion of vinyl acetate-acrylic copolymer resin; emulsion of acrylic acid-vinyl chloride copolymer resin; and emulsion of ethylene-vinyl acetate graft copolymer resin.

As another preferred mode of the invention, the afore-said alkanolamine may be one selected from the group consisting of: monoethanolamine; diethanolamine; triethanolamine; monopropanolamine; dipropanolamine; and tripropanolamine.

Other various features and advantages of the present invention will become apparent from reading of the descriptions hereinafter, with reference to the annexed drawings.

### BRIEF DESCRIPTION OF THE DRAWINGS

FIG. 1 is a ternary composition diagram for showing the mixing ratios (percent by weight) respectively of rubber latex, synthetic resin emulsion and alkanolamine, in a double eyelid forming agent of the present invention.

### DETAILED DESCRIPTION OF PREFERRED EMBODIMENT OF THE INVENTION

In accordance with the present invention, an agent for forming double eyelid in top lid of eye (which shall be simply referred to as “double eyelid forming agent” hereinafter) is essentially comprised of: rubber latex; synthetic resin emulsion; and alkanolamine. Generically stated, such double eyelid forming agent is prepared by mixing those three component elements with one another to provide a mixture and then adding water to that mixture. Hence, the agent per se is a liquid agent.

In the liquid double eyelid forming agent, it is important that the rubber latex used should be of a property not exceeding a statutory permissible range set by the regulations of hazardous materials that will adversely affect human body and natural environment. For example, one example of recommended rubber latex is a low ammonium latex (containing approx. 0.25% of ammonia) with 0.8% or less of total alkali content therein, as prescribed by the code K 6381 of JIS (Japanese Industrial Standards).

Also, it is important that the synthetic resin material used in the double eyelid forming agent be limited to one of appropriate synthetic resin materials to be admixed with the foregoing rubber latex, which fulfils the purposes of: (i) causing the rubber latex to get dry rapidly; (ii) improving water resistance and perspiration resistance (i.e. a property of resisting physiologic saline) which are required for the rubber latex to attain as a resulting film on human eyelid; and (iii) enhancing a rigidity of the film of rubber latex. Further, such synthetic resin material is required to have a good compatibility with afore-defined rubber latex, so that the rubber latex mixed with that particular synthetic resin material is transformed into a film at minimum temperature of 20° C. or less. Also, the synthetic resin material is required to have a sufficient water resistance as well as a sufficient perspiration resistance. Preferably, this sort of synthetic resin material may be an emulsion type of synthetic resin (hereinafter, referred to as “synthetic resin emulsion”) with 38 to 55% by weight of solid content therein. This is however given by way of example and not limitative.

Example of the afore-said synthetic resin emulsion, suited for use in the present invention, includes: emulsion of acrylic ester resin; emulsion of resin of ester of methacrylic acid; emulsion of styrene resin; emulsion of styrene-acrylic copolymer resin; emulsion of styrene-methacrylic copolymer resin; emulsion of acrylamide-acrylamide copolymer resin; emulsion of modified ethylene-vinyl acetate resin; emulsion of ethylene-vinyl acetate copolymer resin; emulsion of vinyl acetate-acrylic copolymer rein; emulsion of acrylic acid-vinyl chloride copolymer resin; and emulsion of ethylene-vinyl acetate graft copolymer resin.

A preferred molecular weight of synthetic resin content in each of the above-listed synthetic resin emulsions may be in the ranges of from 300,000 to 2,000,000, which may of course vary according to what kind of synthetic resin is to be used. It is to be noted here that, if the molecular weight of synthetic resin used is less than 300,000. in the process of preparing a double eyelid forming agent, a resulting film formed from that agent becomes small in elasticity, whereas on the other hand, if the molecular weight of synthetic resin used is increased in excess of 2,000,000, a resulting liquid double eyelid forming agent becomes excessively great in viscosity, which makes that liquid agent less spreadable and therefore a user can hardly apply such liquid agent on the top lid of his or her eye.

Example of the afore-stated alkanolamine suited for use in the present invention includes: monoethanolamine; diethanolamine; triethanolamine; monopropanolamine; dipropanolamine; and tripropanolamine.

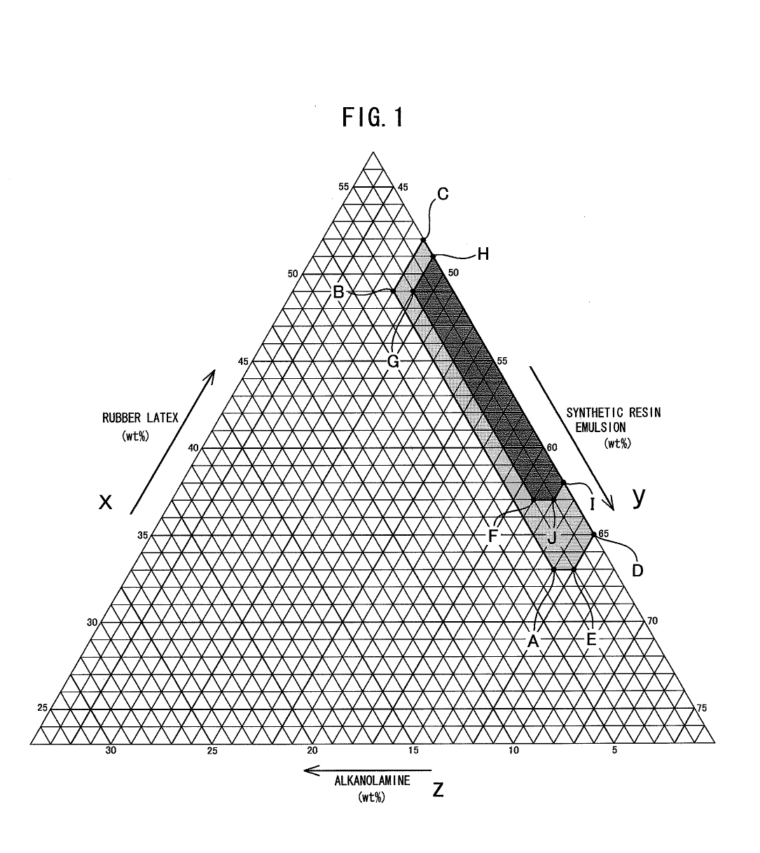

Now, one exemplary mode for preparing the double eyelid forming agent will be described, with reference to Tables 1 and 2 given below, as well as to FIG. 1 of the annexed drawings.

In the present embodiment, a (styrene/alkyl acrylate) copolymer ammonium was used as one example of the aforementioned synthetic resin emulsions, and also, a monoethanolamine was used as one example of the aforementioned alkanolamines. As the rubber latex, the previously stated low ammonium latex defined by JIS was used, which contains approx. 0.25% of ammonia and 0.8% or less of total alkali content. In this connection, the exemplary data given in the Tables 1 and 2 as well as in FIG. 1 were actually obtained by use of those three component elements, but, it is to be noted that, with any one of the previously listed synthetic resin emulsions and alkanolamines, substantially the same data as such exemplary data were obtained. Hence, hereinafter, the three terms, “(styrene/alkyl acrylate) copolymer ammonium”, “monoethanolamine” and “low ammonium latex”, will be omitted for the sake of simplicity, which will instead be generically referred to as “synthetic resin emulsion”, “alkanolamine”, and “rubber latex”, respectively. The same goes for the Tables 1 and 2 as well as for FIG. 1.

In practice, the rubber latex, the synthetic resin emulsion, and the alkanolamine were admixed with one another in accordance with each of different mixing ratios shown in the Tables 1 and 2 to provide a plurality of different mixtures. Thereafter, 5 % by weight of water was added to each of those different mixtures, so that a plurality of liquid agents for forming double eyelid were provided and assigned with the respective sample numbers, as indicated in the Tables 1 and 2. Note that any abbreviated wording “liquid agent” to be used hereinafter refers to the liquid double eyelid forming agent.

Each of the thus-prepared liquid agents was applied on and along a lower end region of top lid of a user's eye and left dry, so that the liquid agent was dried and solidified into a film on that particular region of top lid. Note that the resulting films so formed from the respective liquid agents are in correspondence with the respective sample numbers in the tables.

Thereafter, each of those resulting film samples was analyzed and determined in terms of its appearance, adhesiveness and tensile strength as well as of its storage stability. Result of the analysis for each film sample is shown in the Tables 1 and 2, with the respective overall evaluations added therefor, wherein the symbol “X” indicates that the corresponding agent is “not usable”, wherein the symbol “◯” indicates that the corresponding agent is “effectively usable”, and wherein the symbol “⊚” indicates that the corresponding agent is “most effectively usable”.

Also, in the Tables 1 and 2, the alphabetical letters, “A”, “B”, “C”, “D”, “E”, “F”, “G”, “H”, “I” and “J”., appear in some of the columns titled “Overall evaluation”. Briefly stated, those letters are associated with two different data ranges represented by polygonal (or hexagonal) region shown in FIG. 1 which refer to effective range and most effective range with regard to the aforementioned mixtures of rubber latex, synthetic resin emulsion and alkanolamine.

The letters, “A”, “B”, “C”, “D” and “E”, appearing in the Tables 1 and 2, correspond to the respective designations “A”, “B”, “C”, “D” and “E” in FIG. 1, which constitute the respective five apexes or points of a large hexagonal region, as understandable from the thin hatching. On the other hand, the letters “F”, “G”, “H”, “I” and “J”, appearing in the Tables 1 and 2, correspond to the respective designations “F”, “G”, “H”, “I” and “J” in FIG. 1, which constitute the respective five apexes or points of a small hexagonal region, as understandable from the thick hatching. The details in this respect will be elaborated later on.

TABLE 1

| Sample | Rubber latex | Synthetic resin | Alkanolamine | Appearance | | Tensile | Storage | Overall |
|---|---|---|---|---|---|---|---|---|
| No. | (wt. %) | emulsion (wt. %) | (wt. %) | and color | Adhesiveness | Strength | stability | evaluation |
| 1 | 53 | 47 | 0 | TP: High | Sufficient | Small | Poor | X |
| 2 | 52.9 | 47 | 0.1 | TP: High | Sufficient | Small | Good | X |
| 3 | 52 | 47 | 1 | TP: High | Sufficient | Small | Good | X |
| 4 | 51 | 47 | 2 | TP: High | Sufficient | Small | Good | X |
| 5 | 50 | 47 | 3 | TP: High | Tolerable | Small | Good | X |
| 6 | 49 | 47 | 4 | Slightly white | Insufficient | Small | Good | X |
| 7 | 52 | 48 | 0 | TP: High | Sufficient | Fair | Poor | X |
| 8 | 51.9 | 48 | 0.1 | TP: High | Sufficient | Fair | Good | ◯ C |
| 9 | 51 | 48 | 1 | TP: High | Sufficient | Fair | Good | ◯ |
| 10 | 50 | 48 | 2 | TP: High | Sufficient | Fair | Good | ◯ |
| 11 | 49 | 48 | 3 | TP: High | Tolerable | Fair | Good | ◯ B |
| 12 | 48 | 48 | 4 | Slightly white | Insufficient | Fair | Good | X |
| 13 | 51 | 49 | 0 | TP: High | Sufficient | Great | Poor | X |
| 14 | 50.9 | 49 | 0.1 | TP: High | Sufficient | Great | Good | ◯ H |
| 15 | 50 | 49 | 1 | TP: High | Sufficient | Great | Good | ◯ |
| 16 | 49 | 49 | 2 | TP: High | Sufficient | Great | Good | ◯ G |
| 17 | 48 | 49 | 3 | TP: High | Tolerable | Great | Good | ◯ |
| 18 | 47 | 49 | 4 | Slightly white | Insufficient | Great | Good | X |
| 19 | 50 | 50 | 0 | TP: High | Sufficient | Great | Poor | X |
| 20 | 49.9 | 50 | 0.1 | TP: High | Sufficient | Great | Good | ⊚ |
| 21 | 49 | 50 | 1 | TP: High | Sufficient | Great | Good | ⊚ |
| 22 | 48 | 50 | 2 | TP: High | Sufficient | Great | Good | ⊚ |
| 23 | 47 | 50 | 3 | TP: High | Tolerable | Great | Good | ◯ |
| 24 | 46 | 50 | 4 | Slightly white | Insufficient | Great | Good | X |
| 25 | 45 | 55 | 0 | TP: High | Sufficient | Great | Poor | X |
| 26 | 44.9 | 55 | 0.1 | TP: High | Sufficient | Great | Good | ⊚ |
| 27 | 44 | 55 | 1 | TP: High | Sufficient | Great | Good | ⊚ |
| 28 | 43 | 55 | 2 | TP: High | Sufficient | Great | Good | ⊚ |
| 29 | 42 | 55 | 3 | TP: High | Tolerable | Great | Good | ◯ |
| 30 | 41 | 55 | 4 | Slightly white | Insufficient | Great | Good | X |

Note that the sign “TP” in the column “Appearance” means that the corresponding film sample is transparent and also indicates the degree of transparency thereof.

TABLE 2

| Sample | Rubber latex | Synthetic resin | Alkanolamine | Appearance | | Tensile | Storage | Overall |
|---|---|---|---|---|---|---|---|---|
| No. | (wt. %) | emulsion (wt. %) | (wt. %) | and color | Adhesiveness | Strength | stability | evaluation |
| 31 | 40 | 60 | 0 | TP: High | Sufficient | Great | Poor | X |
| 32 | 39.9 | 60 | 0.1 | TP: High | Sufficient | Great | Good | ⊚ |
| 33 | 39 | 60 | 1 | TP: High | Sufficient | Great | Good | ⊚ |
| 34 | 38 | 60 | 2 | TP: High | Sufficient | Great | Good | ⊚ |
| 35 | 37 | 60 | 3 | TP: Medium | Tolerable | Great | Good | ◯ |
| 36 | 36 | 60 | 4 | Slightly white | Insufficient | Great | Good | X |
| 37 | 39 | 61 | 0 | TP: High | Sufficient | Great | Poor | X |
| 38 | 38.9 | 61 | 0.1 | TP: High | Sufficient | Great | Good | ⊚ |
| 39 | 38 | 61 | 1 | TP: High | Sufficient | Great | Good | ⊚ |
| 40 | 37 | 61 | 2 | TP: High | Sufficient | Great | Good | ⊚ F |
| 41 | 36 | 61 | 3 | TP: Medium | Tolerable | Great | Good | ◯ |
| 42 | 35 | 61 | 4 | Slightly white | Insufficient | Great | Good | X |
| 43 | 38 | 62 | 0 | TP: High | Sufficient | Great | Poor | X |
| 44 | 37.9 | 62 | 0.1 | TP: High | Sufficient | Great | Good | ⊚ I |
| 45 | 37 | 62 | 1 | TP: High | Sufficient | Great | Good | ⊚ J |
| 46 | 36 | 62 | 2 | TP: Medium | Tolerable | Great | Good | ◯ |
| 47 | 35 | 62 | 3 | TP: Medium | Tolerable | Great | Good | ◯ |
| 48 | 34 | 62 | 4 | Slightly white | Insufficient | Great | Good | X |
| 49 | 37 | 63 | 0 | TP: Medium | Sufficient | Great | Poor | X |
| 50 | 36.9 | 63 | 0.1 | TP: Medium | Sufficient | Great | Good | ◯ |
| 51 | 36 | 63 | 1 | TP: Medium | Tolerable | Great | Good | ◯ |
| 52 | 35 | 63 | 2 | TP: Medium | Tolerable | Great | Good | ◯ |
| 53 | 34 | 63 | 3 | TP: Medium | Tolerable | Great | Good | ◯ |
| 54 | 33 | 63 | 4 | Slightly white | Insufficient | Great | Good | X |
| 55 | 36 | 64 | 0 | TP: Medium | Tolerable | Great | Poor | X |
| 56 | 35.9 | 64 | 0.1 | TP: Medium | Tolerable | Great | Good | ◯ |
| 57 | 35 | 64 | 1 | TP: Medium | Tolerable | Great | Good | ◯ |
| 58 | 34 | 64 | 2 | TP: Medium | Tolerable | Great | Good | ◯ |
| 59 | 33 | 64 | 3 | TP: Medium | Tolerable | Great | Good | ◯ A |
| 60 | 32 | 64 | 4 | Slightly white | Insufficient | Great | Good | X |
| 61 | 35 | 65 | 0 | TP: Medium | Tolerable | Great | Poor | X |
| 62 | 34.9 | 65 | 0.1 | TP: Medium | Tolerable | Great | Good | ◯ D |
| 63 | 34 | 65 | 1 | TP: Medium | Tolerable | Great | Good | ◯ |
| 64 | 33 | 65 | 2 | TP: Medium | Tolerable | Great | Good | ◯ E |
| 65 | 32 | 65 | 3 | Slightly white | Insufficient | Great | Good | X |
| 66 | 31 | 65 | 4 | White | Insufficient | Great | Good | X |
| 67 | 34 | 66 | 0 | White | Insufficient | Great | Poor | X |
| 68 | 33.9 | 66 | 0.1 | White | Insufficient | Great | Good | X |
| 69 | 33 | 66 | 1 | White | Insufficient | Great | Good | X |
| 70 | 32 | 66 | 2 | White | Insufficient | Great | Good | X |
| 71 | 31 | 66 | 3 | White | Insufficient | Great | Good | X |
| 72 | 30 | 66 | 4 | White | Insufficient | Great | Good | X |

Note that the sign “TP” in the column “Appearance” means that the corresponding film sample is transparent and also indicates the degree of transparency thereof.

The ternary composition diagram in FIG. 1 is plotted on the basis of the foregoing data of experiments, according to the mixing ratios of samples in the Tables 1 and 2 with respect to the rubber latex (at x-axis), synthetic resin emulsion (at y-axis) and alkanolamine (at z-axis). The three arrows in the FIG. 1 indicate the respective increasing directions of mixing ratios of such three component elements.

Reference is now made to the sample Nos. 1 to 6 in the Table 1 above, from which it is observed that each of the corresponding film samples contains 47% by weight of the synthetic resin emulsion and is small in tensile strength. In this regard, a comparative view of the FIG. 1 and Table 1 indicates that a line extending between two points B and C in the FIG. 1 is given for showing effective ranges of mixing ratios respectively of the rubber latex, synthetic resin emulsion and alkanolamine (hereinafter, “the three component elements”, for simplicity) in reference to 48% by weight or more of the synthetic resin emulsion, in conjunction with the corresponding symbols “◯” given in the Table 1. It is to be seen therefrom that the mixing ratio of synthetic resin emulsion in original liquid agent prepared, which falls in a region above such line between B and C in the ternary diagram, is smaller than 48% by weight, and in that case, a resulting film formed from the agent is small in tensile strength. Namely, the tensile strength of the film is insufficient in tensile strength to overcome an excessively high tautness of top lid skin of user's eye that has been discussed previously. Experiments shows that the lower end region of top lid on which the film is secured did not become so hard as to cause a fold to create along the film in the top lid. Consequently, in the present case, it was concluded that a double eyelid can hardly be formed in the excessively taut skin of top lid.

By contrast, with regard to the sample Nos. 8 to 11 wherein the corresponding film samples each contains 48% by weight of synthetic resin emulsion, a tensile strength of each of the film samples is found great. Namely, referring again to the aforementioned line between B and C and from a comparative view of the FIG. 1 and Table 1, it is to be seen that the mixing ratio of synthetic resin emulsion in original liquid agent prepared, which falls in a region below but inclusive of the line between B and C in the ternary diagram, is not lower than 48% by weight, and therefore a resulting film formed from the agent is of sufficient tensile strength to overcome the excessively high tautness of top lid skin that has been discussed previously. Experiments show that the region of top lid on which the film is secured was hardened enough to cause a fold to create along the film in the top lid. Thus, in the present case, it was concluded that a double eyelid can be formed in the excessively taut skin of top lid.

The sample Nos. 1, 7, 13, 19, 25, 31, 37, 43, 49, 55, 61 and 67 given in Tables 1 and 2 each contains 0% by weight of alkanolamine. In those cases, no alkanolamine was admixed with rubber latex and synthetic resin emulsion in the process of preparing original liquid agent, and therefore, experiments show that any of the liquid agents with no alkanolamine added thereto was poor in storage stability and did not maintain its quality for a long period of time, and that some of the liquid agents become solid during a lengthy period of storage time. Further, experiments show that such liquid agent with no alkanolamine therein was hardly transformed into a required state of film, and that even a resulting film of the agent was insufficient in water resistance.

On the other hand, the samples Nos. 2, 8, 14, 20, 26, 32, 38, 44, 50, 56, 62 and 68 each contains 0.1% by weight of alkanolamine, in which case, the corresponding film samples are sufficient in storage stability. In this connection, referring to FIG. 1 as well as to Tables 1 and 2, a line extending between two points C and D in the FIG. 1 indicates effective ranges of mixing ratios respectively of the three component elements in reference to 0.1% by weight or more of the alkanolamine, in conjunction with the corresponding symbols “◯” in the Tables 1 and 2. It is to be seen therefrom that the mixing ratio of alkanolamine in original liquid agent prepared, which falls in a region inwardly of the ternary diagram from such line between C and D, is not lower than 0.1% by weight. In that case, any of the corresponding liquid agents is good in storage stability. Experiments show that all the liquid agents prepared under such conditions did not become sold even for a lengthy period of storage time.

In this context, the samples Nos. 6, 12, 18, 24, 30, 36, 42, 48, 54, 60, 66 and 72 each contains 4% by weight of alkanolamine, in which case, none of the corresponding film samples attains required adhesiveness. In this connection, as understandable from FIG. 1 as well as Tables 1 and 2, a line extending between two points A and B in the FIG. 1 indicates effective ranges of mixing ratios of the three component elements in reference to lower than 4% by weight of alkanolamine, in conjunction with the corresponding symbols “◯” in Tables 1 and 2. It is to be seen therefrom that a mixing ratio of the alkanolamine in original liquid agent prepared, which falls in a region inwardly of the ternary diagram from the line between A and B, is in excess of 4% by weight. In that case, any resulting film formed from the agent is insufficient in adhesiveness. Namely, experiments show that the film did not adhere well to the excessively high tautness of skin of user's top lid, and in particular, when exposed to water such as sweat, the film was easily broken in pieces and easily removed from the skin of top lid. It was therefore concluded that a stable double eyelid can hardly be formed in the top lid in the present instance.

In contrast thereto, the sample Nos. 5, 11, 17, 23, 29, 35, 41, 47, 53, 59, 65 and 71 each contains 3% by weight of alkanolamine, in which case, adhesiveness of each of the corresponding film samples is tolerably sufficient. Referring again to the aforementioned line between A and B, and from comparative view of FIG. 1 and Tables 1 and 2, it is to be seen that the mixing ratio of alkanolamine in original liquid agent prepared, which falls within the thin hatched polygonal region with respect to the line between A and B, is not larger than 3% by weight. In that case, any resulting film formed from the agent sufficiently adheres to the excessively taut skin of top lid and provides a satisfied resistance to water such as sweat. It was therefore concluded that a stable double eyelid can be formed in the top lid in the present instance.

The sample No. 65 contains 65% by weight of synthetic resin emulsion, and the sample Nos. 67 to 72 each contains 66% by weight of synthetic resin emulsion. In those cases, all the corresponding film samples become white in color and appearance. In this connection, reference is particularly made to a first line extending between two points D and E as well as to a second line extending between two points A and E, in FIG. 1. A comparative view of Table 2 and FIG. 1 indicates that the first and second lines show effective ranges of mixing ratios of the three component elements in reference to less than 65% by weight of synthetic resin emulsion, in conjunction with the corresponding symbols “◯” in the Table 2. In the present case, all the mixing ratios of corresponding synthetic resin emulsions were out of the thin hatched polygonal region as well as from the foregoing first and second lines, which means that all the mixing ratios of synthetic resin emulsions exceeded 65% by weight. All the resulting films in the present case were white in color and appearance, and experiments show that creases, wrinkles and cracks were easily created in most of such white films. It was therefore concluded that a double eyelid can hardly be formed in the top lid.

In contrast thereto, the sample Nos. 62, 63 and 64 each contains 65% by weight of synthetic resin emulsion. In this case, each of the corresponding film samples was slightly white and cloudy in appearance, but maintained a required degree of transparency, as evaluated by “medium” in transparency in Table 2. Referring again to the aforementioned first line between D and E and second line between A and E, and also from comparative view of FIG. 1 and Table 2, it is to be seen that any mixing ratio of synthetic resin emulsion in original liquid agent prepared, which falls within the thin-hatched polygonal region from either of the first and second limit lines, is not larger than 64% by weight, or not larger than 65% by weight. In that case, a resulting film formed from such agent is slightly white and cloudy in appearance, but maintains a required degree of transparency. Experiments show that neither of crease, wrinkle and crack was found in the film. It was therefore concluded that all the liquid agents in the present case are usable effectively for forming a double eyelid in the top lid.

In this context, reference is made to FIG. 1 in which the ternary composition diagram is illustrated with three coordinate axes x, y and z, which indicates mixing ratios for the respective foregoing three component elements in ternary correlated manner, wherein the x-axis relates to mixing ratios (wt. %) for the rubber latex, the y-axis relates to mixing ratios (wt. %) for the synthetic resin emulsion, and the z-axis relates to mixing ratios (wt. %) for the alkanolamine. In such ternary composition diagram, based upon the above-described data and symbols “◯” in Tables 1 and 2, a first polygonal (or hexagonal) region was plotted in relation to those three coordinate axes x, y and z, which is indicated by the thin hatched area in FIG. 1. It is observed that the following five points A, B, C, D and E are defined as five respective apexes of such first polygonal region.
A (33.0, 64.0, 3.0)
B (49.0, 48.0, 3.0)
C (51.9, 48.0, 0.1)
D (34.9, 65.0, 0.1)
E (33.0, 65.0, 2.0)

Accordingly, insofar as the mixing ratios of rubber latex, synthetic resin emulsion and alkanolamine fall within the above-plotted first polygonal region having the apexes A, B, C, D and E, any one of the correspondingly prepared agents can be effectively used for forming double eyelid in an excessively taut skin of top lid of user's eye. Namely, any resulting film formed from the agent achieves a required degree for its transparency, adhesiveness, tensile strength and storage stability, so that a double eyelid can be effectively formed in the top lid.

Referring o the Table 1 and FIG. 1, the sample Nos. 14 to 17 each contains 49% by weight of synthetic resin emulsion. In this instance, the tensile strength of each of the corresponding film samples was sufficient. Namely, referring in particular to a line extending between two points G and H in FIG. 1, it is to be seen that, insofar as the mixing ratio of synthetic resin emulsion in original liquid agent prepared falls within the thick hatched polygonal region from that line between G and H, any resulting film formed from the agent has a sufficient tensile strength to overcome the above-mentioned excessively high tautness of top lid. Accordingly, a stable double eyelid can be formed in the excessively taut skin of top lid in the present instance.

With reference to the Table 2 and FIG. 1, the sample No. 40 contains 61% by weight of synthetic resin emulsion, and the sample Nos. 44 and 45 each contains 62% by weight of synthetic resin emulsion. In this instance, the transparency of each of the corresponding film samples was high. Reference is particularly made to one line extending between two points and I and J as well as to another line extending between two points J and F, in FIG. 1. It is to be seen therefrom that, insofar as the mixing ratio of synthetic resin emulsion in original liquid agent prepared falls within the thick hatched polygonal region from each of those two lines, any resulting film formed from the agent is high in transparency. It was therefore concluded that a highly transparent film is attainable in the present case, so that the film itself is almost invisible and not recognizable by other person. Hence, a double eyelid can be formed in natural way, without objectionable point, in the excessively taut skin of top lid.

With reference to the Tables 1 and 2 as well as to FIG. 1, the sample Nos. 4, 10, 16, 22, 28, 34, 40, 46, 52, 58, 64 and 70 each contains 2.0% by weight of alkanolamine. In this instance, the adhesiveness of each of the corresponding film samples was great and sufficient. In this respect, particular reference is made to a line extending between two points F and G in FIG. 1. It is to be seen therefrom that, insofar as the mixing ratio of alkanolamine in original liquid agent prepared falls within the thick hatched polygonal region from such line between F and G, any resulting film formed from the agent has a highly improved water resistance, and experiments show that adherence of the film to the excessively taut top lid was far more improved in comparison with other film samples.

Based upon all the data evaluated “most effectively usable” as indicated by symbols “⊚” in Tables 1 and 2, and with the above-described aspects in view, a second polygonal (or hexagonal) region was plotted in relation to the three coordinate axes x, y and z in the ternary composition diagram in FIG. 1. In the present case, the thick hatched area in that diagram indicates such particular second polygonal region, from which it is observed that the following five points F, G, H, I and J are defined as five respective apexes of the second polygonal region.
F (37.0, 61.0, 2.0)
G (49.0, 49.0, 2.0)
H (50.9, 49.0, 0.1)
I (37.9, 52.0, 0.1)
J (37.0, 62.0, 1.0)

Accordingly, insofar as the mixing ratios of rubber latex, synthetic resin emulsion and alkanolamine fall within the above-plotted second polygonal region with the apexes F, G, H, I and J, any one of the correspondingly prepared agents achieves exceptionally remarkable effects for forming double eyelid in the excessively taut skin of top lid. Namely, any resulting film formed from such agent achieves excellent degree for all of its transparency, adhesiveness, tensile strength and storage stability, in comparison with the films associated with the earlier stated first polygonal region. Hence, a double eyelid can be most effectively formed in the top lid.

From the descriptions above, it is to be appreciated that the present invention has the following effects and advantages:

(i) The liquid double eyelid forming agent in the present invention contains an increased mixing ratio of the synthetic resin emulsion relative to the conventional mixing ratio of synthetic resin emulsion (15 to 50% by weight) given in. the JP 6-62384 stated earlier. Therefore, in accordance with the present invention, the liquid agent with such increased mixing ratio of synthetic resin emulsion effectively becomes a strong film of sufficient tensile strength for overcoming an excessively high tautness of skin of top lid, thereby insuring stable formation of double eyelid in that taut top lid.

(ii) Also, according to the present invention, the mixing ratio of rubber latex contained in the liquid double eyelid forming agent is made small relative to the conventional mixing ratio of rubber latex (85% to 50% by weight) given in the foregoing JP 6-62384. Thus, a resulting film created in the present invention has non-shine surface and is superior in transparency to a resulting film of that Japanese prior art. Accordingly, in accordance with the present invention, the film itself is almost invisible and not recognizable by other person.

In this context, it is also to be appreciated that the present invention is provided by improving the foregoing JP 6-62384 to fulfil the purpose of realizing stable formation of double eyelid in the taut skin of top lid.

While having described the present invention thus far, it should be understood that the invention is not limited to the illustrated embodiment, but, any modification, replacement and addition may be applied thereto without departing from the scopes of the appended claims. For example, the following elements: ammonia water; preservative; pigment; perfume and water, may alone or in combination be added to the above-described double eyelid forming agent in a proper amount that does not deteriorate any of the properties and features stated above.

What is claimed is:

1. An agent for forming double eyelid, which is characterized by containing:
rubber latex;
synthetic resin emulsion; and
alkanolamine,
said rubber latex, said synthetic resin emulsion and said alkanolamine being admixed with one another, based on weight percent, such that mixing ratios respectively of said rubber latex, said synthetic resin emulsion and said alkanolamine fall within a polygonal region with five apexes A, B, C, D and E, said polygonal region being defined in a ternary composition diagram plotted with three coordinate axes x, y and z as shown in FIG. 1, wherein said x relates to mixing ratios (percent by weight) of said rubber latex, said y relates to mixing ratios (percent by weight) of said synthetic resin emulsion, and said z relates to mixing ratios (percent by weight) of alkanolamine, and wherein said five apexes A, B, C, D and E are defined as follows in relation to said x, said y and said z.
A (33.0, 64.0, 3.0)
B (49.0, 48.0, 3.0)
C (51.9, 48.0, 0.1)
D (34.9, 65.0, 0.1)
E (33.0, 65.0, 2.0)

2. The agent as claimed in claim 1, wherein said synthetic resin emulsion is one selected from the group consisting of: emulsion of acrylic ester resin; emulsion of resin of ester of methacrylic acid; emulsion of styrene resin; emulsion of styrene-acrylic copolymer resin; emulsion of styrene-methacrylic copolymer resin; emulsion of acrylamide-acrylamide copolymer resin; emulsion of modified ethylene-vinyl acetate copolymer resin; emulsion of ethylene-vinyl acetate copolymer resin; emulsion of vinyl acetate-acrylic copolymer resin; emulsion of acrylic acid-vinyl chloride copolymer resin; and emulsion of ethylene-vinyl acetate graft copolymer resin.

3. The agent as claimed in claim 1, wherein a molecular weight of synthetic resin in said synthetic resin emulsion is in a range of from 300,000 to 2,000,000.

4. The agent as claimed in claim 1, wherein said alkanolamine is one selected from the group consisting of: monoethanolamine; diethanolamine; triethanolamine; monopropanolamine; dipropanolamine; and tripropanolamine.

5. An agent for forming double eyelid, which is characterized by containing:
rubber latex;
synthetic resin emulsion; and
alkanolamine,
said rubber latex, said synthetic resin emulsion and said alkanolamine being admixed with one another, based on weight percent, such that mixing ratios respectively of said rubber latex, said synthetic resin emulsion and said alkanolamine fall within a polygonal region with five apexes F, G, H, I and J, said polygonal region being defined in a ternary composition diagram plotted with three coordinate axes x, y and z as shown in FIG. 1, wherein said x relates to mixing ratios (percent by weight) of said rubber latex, said y relates to mixing ratios (percent by weight) of said synthetic resin emulsion, and said z relates to mixing ratios (percent by weight) of alkanolamine, and wherein said five apexes F, G, H, I and J are defined as follows in relation to said x, said y and said z.
F (37.0, 61.0, 2.0)
G (49.0, 49.0, 2.0)
H (50.9, 49.0, 0.1)
I (37.9, 62.0, 0.1)
J (37.0, 62.0, 1.0)

6. The agent as claimed in claim 5, wherein said synthetic resin emulsion is one selected from the group consisting of: emulsion of acrylic ester resin; emulsion of resin of ester of methacrylic acid; emulsion of styrene resin; emulsion of styrene-acrylic copolymer resin; emulsion of styrene-methacrylic copolymer resin; emulsion of acrylamide-acrylamide copolymer resin; emulsion of modified ethylene-vinyl acetate copolymer resin; emulsion of ethylene-vinyl acetate copolymer resin; emulsion of vinyl acetate-acrylic copolymer resin; emulsion of acrylic acid-vinyl chloride copolymer resin; and emulsion of ethylene-vinyl acetate graft copolymer resin.

7. The agent as claimed in claim 5, wherein a molecular weight of synthetic resin in said synthetic resin emulsion is in a range of from 300,000 to 2,000,000.

8. The agent as claimed in claim 5, wherein said alkanolamine is one selected from the group consisting of: monoethanolamine; diethanolamine; triethanolamine; monopropanolamine; dipropanolamine; and tripropanolamine.
